(19) United States
(12) Patent Application Publication
(10) Pub. No.: US 2014/0155757 A1
(43) Pub. Date: Jun. 5, 2014

(54) PORTABLE EXOSCOPE IMAGING DEVICE FOR THE HUMAN BODY

(71) Applicants: Young Chul Yang, Seoul (KR); Wan Suk Yang, Seoul (KR)

(72) Inventors: Young Chul Yang, Seoul (KR); Wan Suk Yang, Seoul (KR)

(21) Appl. No.: 14/233,916

(22) PCT Filed: Aug. 31, 2011
(86) PCT No.: PCT/KR2011/006429

§ 371 Date: Jan. 21, 2014

(30) Foreign Application Priority Data
Jul. 22, 2011 (KR) 10-2011-0073007

(51) Int. Cl. A61B 5/00; A61B 1/06; A61B 1/07; A61B 1/05

(52) U.S. Cl. CPC A61B 5/0084; A61B 1/051; A61B 1/0684; A61B 1/07
USPC 600/476

(57) ABSTRACT

The present invention relates to a portable CCD camera imaging device for the human body, comprising: a handle-shaped main body (10); a tube body (20) projecting from the leading end of the main body (10) at an angle to the main body (10) and having a diameter that enables the insertion thereof into a hole in a human body; a CCD camera (30) inserted into the inner diameter of the tube body (20); LEDs (40) installed around the perimeter of the CCD camera (30); and a transmitting ring (50) transmitting light from the LEDs (40) at a uniform brightness, and having a convex portion (52) for diffusing the transmitted light.

### TECHNICAL FIELD

This invention relates to a skin imaging system for the human body that is designed to be small and portable. The system includes: a main body shaped to be easily gripped by the hand; a pipe-line body formed on the top of the main body, for putting inside the cavity of the human body, where the lengthwise axis of the pipe-line body is inclined against that of the main body and the tip is protruded in the lengthwise direction from the main body; a charge coupled device (CCD) camera module installed inside the pipe-line body; LEDs installed sparsely around the CCD camera module; and a light transmission material ring with a convex portion for uniformly diffusing the light from the LEDs.

### BACKGROUND ART

Conventional small camera devices are used in the field of endoscopes. Imaging technology related to conventional camera devices was disclosed in Korean Utility Model Registration No. 20-0361097 and Korean Patent Publication No. 10-2010-0124533. Technology of small imaging devices adjusting the focus was also disclosed in Korean Utility Model Registration No. 20-0416151 and Korean Patent Publication No. 10-2004-0046759.

Conventional endoscopes are developed to examine the interior of a hollow organ or cavity of the body. Conventional common imaging devices are also developed to view the skin of the body or the scalp. These conventional devices do not allow an individual to correctly view the organs that he/she wants to view, e.g., the cavities of the nose and ears, the portions near the eyes, the inside of the mouth, the anus, etc. However, the portable skin imaging system according to the invention is designed so that an individual can view the organs that he/she could not view with his/her naked eye. The system employed in the fields of ophthalmology, dental service, otorhinolaryngology, urology, etc., can examine diseases at the initial step.

### DISCLOSURE

### Technical Problem

The invention has been made in view of the above problems, and provides a portable skin imaging system that includes: a CCD camera module that is installed into the pipe-line body inclined and formed at the top of the main body, and a pipe-line body that can be inserted in the cavity of the human body, unlike a conventional endoscope that has to have a correct diameter to be inserted in the cavity of the human body and is inserted in the interior of a hollow organ of the human body, so that it can easily acquire the inner cavity video of the human body via the camera module.

The invention further provides a portable skin imaging system configured in such a way that, when it is inserted in and examines the cavity of the human body, LEDs can uniformly emit light via the pattern layer and accordingly the CCD camera module can acquire the inner cavity video with uniform illuminance, thereby providing the inner cavity video without reflective light.

The invention further provides a portable skin imaging system configured in such a way that: LEDs are sparsely installed forming a circle; and a light transmission material ring is installed to the front of the LEDs, where the light transmission material ring has a convex portion on the surface, the center axis of a lens module is consistent with the axis of the light transmission material ring, the lens module receives the light from the LEDs, and the outer periphery of the lens module is designed to provide uniform illuminance of light from the LEDs, compared with the center portion of the lens module.

### Technical Solution

In accordance with an exemplary embodiment of the invention, the invention provides a portable skin imaging system configured in such a way that: the main body includes a pipe-line body that is integrally formed therewith and has a diameter appropriate to be inserted into the cavity of the human body; the pipe-line body includes a lens module and a light transmission material ring in the inside; the light transmission material ring provides uniform light to the cavity of the human body; and a CCD camera module acquires the inner cavity video with uniform illuminance.

In accordance with another exemplary embodiment of the invention, the invention provides a portable skin imaging system for the human body, including: a main body shaped to be easily gripped by the hand; a pipe-like body, formed on the top of the main body, for putting in the cavity of the human body, the lengthwise axis being inclined against that of the main body, and the tip being protruded in the lengthwise direction from the main body; a charge coupled device (CCD) camera module installed inside the pipe-line body; LEDs installed sparsely around the CCD camera module; and a light transmission material ring with a convex portion for uniformly diffusing the light from the LEDs.

Preferably, the CCD camera module includes: a lens module shaped a pipe, for adjusting the focus; and a CCD chip installed inside the lens module, for reading video data.

Preferably, the lens module forms a guide groove on the outer surface in the lengthwise direction. The guide groove guides the movement of a guide protrudently formed on the inner wall. The lens module also forms a spur rack gear for transferring movement power on the outer surface.

Preferably, the spur rack gear is engaged with spur gears one of which is partially exposed from the pipe-line body or the main body and rotated by the user's finger. The focus of the lens module may be controlled by an electric motor. It may also be controlled by the attractive or repulsive principle, e.g., an attractive or repulsive force by an electro-magnet.

### Advantageous Effects

As described above, since the portable skin imaging system according to the invention is configured in such a way that a CCD camera module is installed into the pipe-line body inclined and formed at the top of the main body, and a pipe-line body is inserted in the cavity of the human body, unlike a conventional endoscope that has a correct diameter to be inserted in the cavity of the human body and is inserted in the interior of a hollow organ of the human body, it can easily acquire the inner cavity video of the human body via the camera module.

Since the portable skin imaging system is configured in such a way that, when it is inserted in and examines the cavity of the human body, LEDs can uniformly emit light via the pattern layer and accordingly the CCD camera module can acquire the inner cavity video with uniform illuminance, it can provide the inner cavity video without reflective light.

The portable skin imaging system is configured in such a way that: LEDs are sparsely installed forming a circle; and a light transmission material ring is installed in front of the LEDs, where the light transmission material ring has a convex portion on the surface, the center axis of a lens module is consistent with the axis of the light transmission material ring, the lens module receives the light from the LEDs, and the outer periphery of the lens module is designed to uniformly illuminate light from the LEDs, compared with the center portion of the lens module.

### DESCRIPTION OF DRAWINGS

The features and advantages of the present invention will become more apparent from the following detailed description in conjunction with the accompanying drawings, in which

FIG. 1 illustrates a front view of a portable skin imaging system according to an embodiment of the invention;

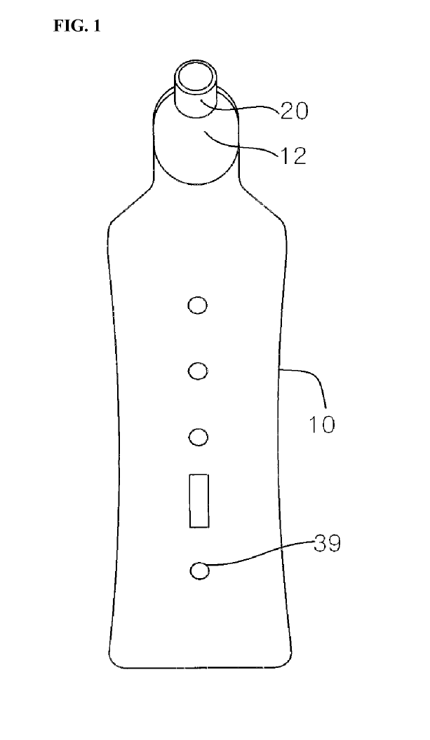

FIG. 2 illustrates a side view of the portable skin imaging system shown in FIG. 1;

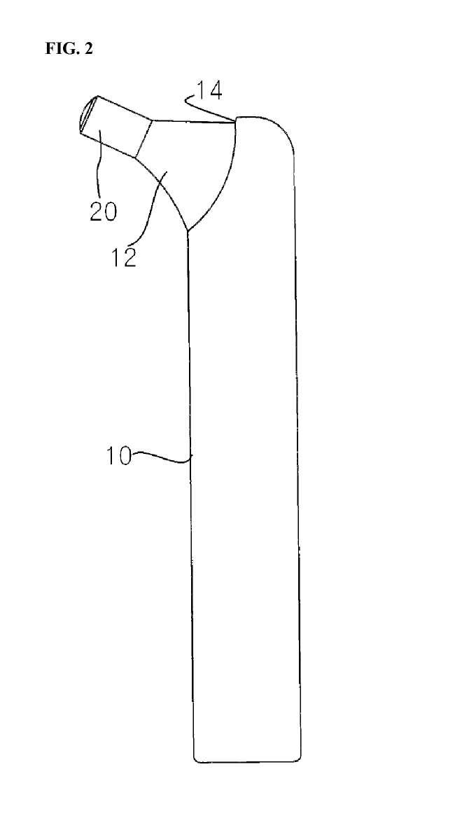

FIG. 3 illustrates a cross-sectional side view of a part of the portable skin imaging system shown in FIG. 2;

FIG. 4 illustrates a cross-sectional view along line A-A shown in FIG. 3;

FIG. 5 illustrates an enlarged cross-sectional side view of a primary part of the portable skin imaging system;

FIG. 6 illustrates a front view of the light transmission material ring shown in FIG. 5;

FIG. 7 illustrates a schematic block diagram of a control block in the portable skin imaging system according to an embodiment of the invention;

FIG. 8 illustrates a control box of the portable skin imaging system according to an embodiment of the invention; and

FIG. 9 illustrates a cross-sectional view of the control box shown in FIG. 8.

### BEST MODE

The invention provides a portable skin imaging system including: a main body shaped to be easily gripped by the hand; a pipe-like body with a certain diameter, formed on the top of the main body, for putting inside the cavity of the human body, the lengthwise axis being inclined against that of the main body, and the tip being protruded in the lengthwise direction from the main body; a charge coupled device (CCD) camera module installed inside the pipe-line body; LEDs installed sparsely around the CCD camera module; and a light transmission material ring with a convex portion for uniformly diffusing the light from the LEDs.

### Mode for Invention Hereinafter, exemplary embodiments of the invention are described in detail with reference to the accompanying drawings.

The portable skin imaging system is configured to include: a main body 10 shaped to be easily gripped by the hand; a pipe-like body 20, formed on the top of the main body 10, for putting inside the cavity of the human body, the lengthwise axis being inclined against that of the main body 10, and the tip being protruded in the lengthwise direction from the main body 10; a charge coupled device (CCD) camera module 30 installed inside the pipe-line body 20; LEDs 40 installed sparsely around the CCD camera module 30; and a light transmission material ring 50 with a convex portion 52 for uniformly diffusing the light from the LEDs 40.

The pipe-line body 20 includes a truncated cone-shape portion 12 the bottom size of which increase in the diameter toward the main body 10 and a stopper step 14 for stopping the insertion into a cavity of the human body.

The light transmission material ring 50 is fixed to the front top of the pipe-line body 20 so that it can be located at the front end of the lens module of the CCD camera module 30. Fixing the light transmission material ring 50 to the pipe-line body 20 may be implemented with a bonding means, screwing coupling, a hooking coupling, etc.

The light transmission material ring 50 forms, in the inside, a pattern layer 53 for limiting the light transmission of light emitted from the LEDS 40. The pattern layer 53 increases larger patterns 51 in parts where the LEDs 40 exist and decreases them in parts where the LEDs 40 do not exist. The light transmission material ring 50 forms, in the front, a convex portion 52 for diffusing light from the LEDs 40 in the radial direction.

The CCD camera module 30 includes: a lens module (32) shaped like a pipe, for adjusting the focus; and a CCD chip 34 installed inside the lens module 32, for reading video data.

The lens module 32 forms a guide groove 33 on the outer surface in the lengthwise direction. The guide groove 33 guides the movement of a guide 24 protrudently formed on the inner wall 22. The lens module 32 also forms, on the outer surface, a spur rack gear 26 for transferring movement power.

The spur rack gear 26 is engaged with spur gears 28 one of which is partially exposed from the pipe-line body 20 or the main body 10 and rotated by the user's finger. Although the embodiment is implemented with the spur gear 26 and spur gears 28, it should be understood that the invention is not limited to the embodiment.

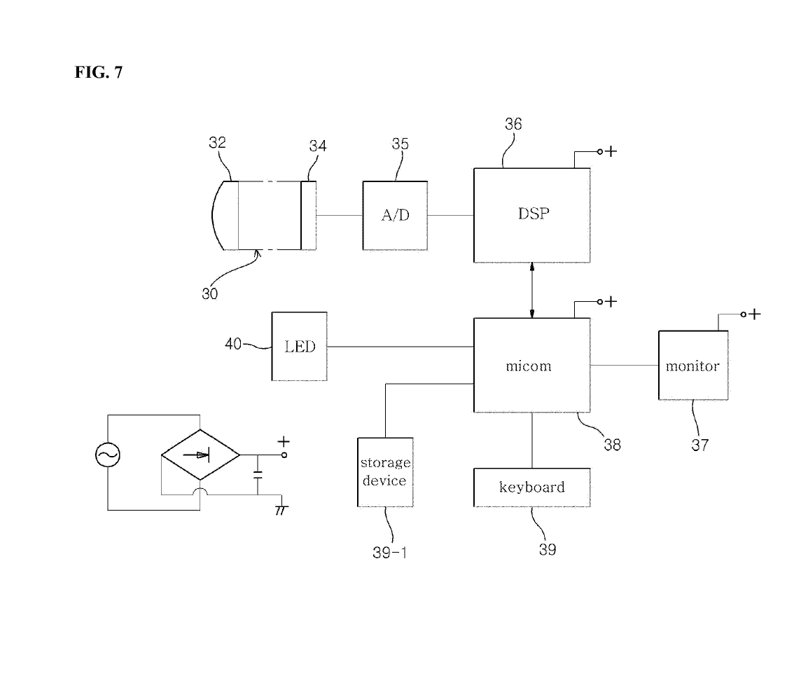

FIG. 7 illustrates a schematic block diagram of a control block in the portable skin imaging system according to an embodiment of the invention. The control block includes: a CCD camera module 30 with a lens module 32 and a CCD chip 34; an analog-to-digital (A/D) converter 35 for converting video data acquired via the CCD chip 34 into digital data; a digital signal processor (DSP) 36 for processing the digital data from the AD converter 35 in the standard format to be displayed on a monitor 3; a micom 38 for controlling the DSP 36; a keyboard 39 for creating signals to control the micom 38; and a storage device 39-1 connected to an output port P2 of the micom 38, for storing programs and data.

The micom 38 drives the LEDs 40 via another output port P1. The components in the portable skin imaging system are operated via electric power supplied by a battery or an adapter that converts AC to DC power. Although a power switch is not shown in the drawings, it should be understood that the embodiment includes it.

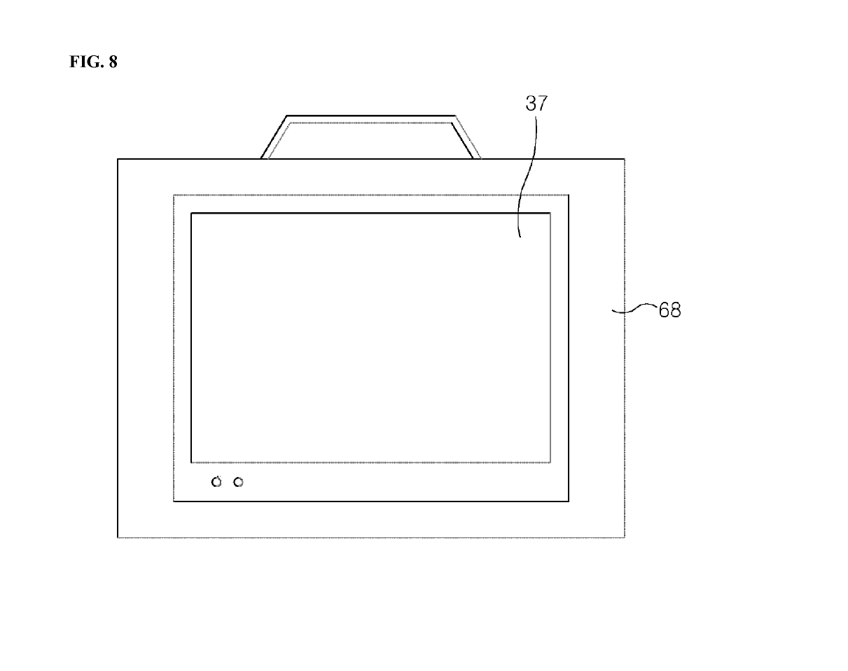

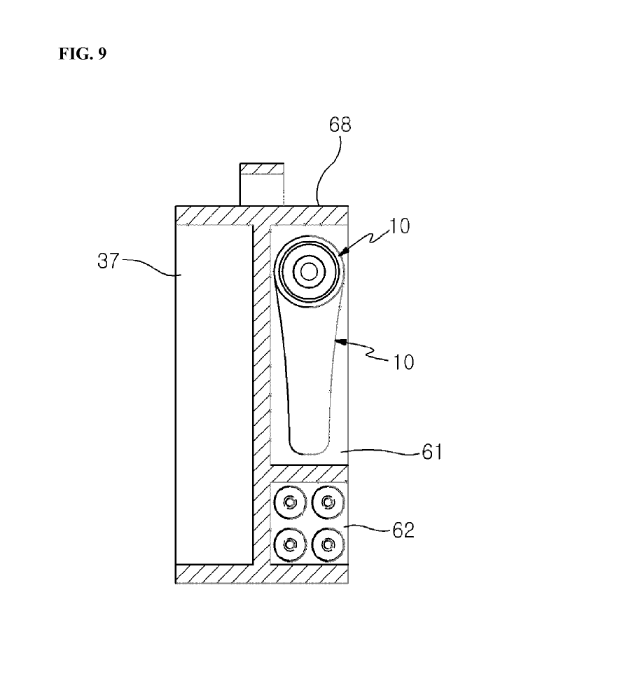

FIG. 8 illustrates a control box of the portable skin imaging system according to an embodiment of the invention, and FIG. 9 illustrates a cross-sectional view of the control box shown in FIG. 8. The control box 60 includes a monitor 37 on one side, a compartment 61 for keeping the main body 10 on another side, a compartment 62 for keeping the power supply, and a handle 63 on the top.

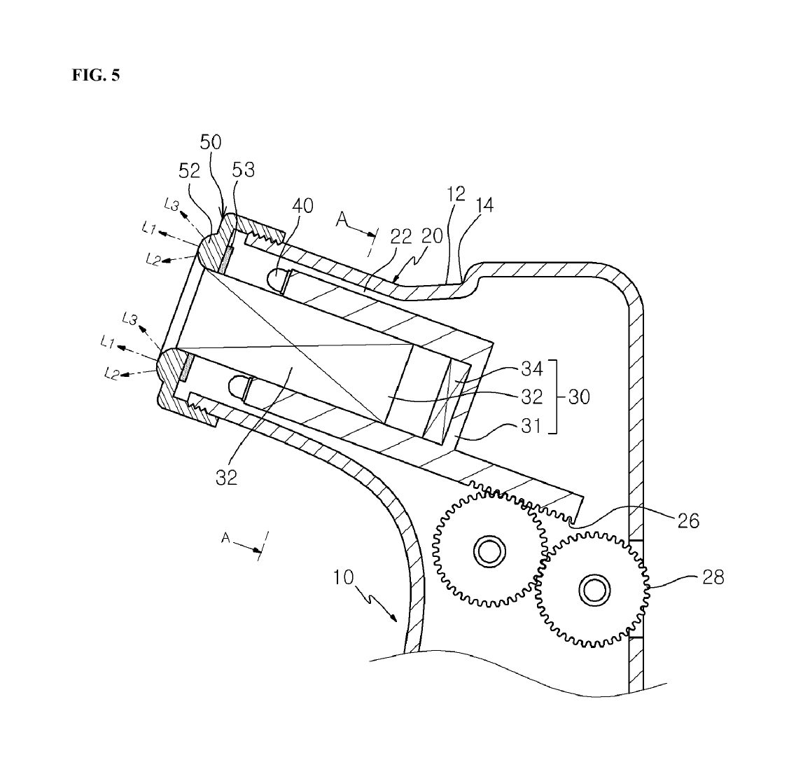

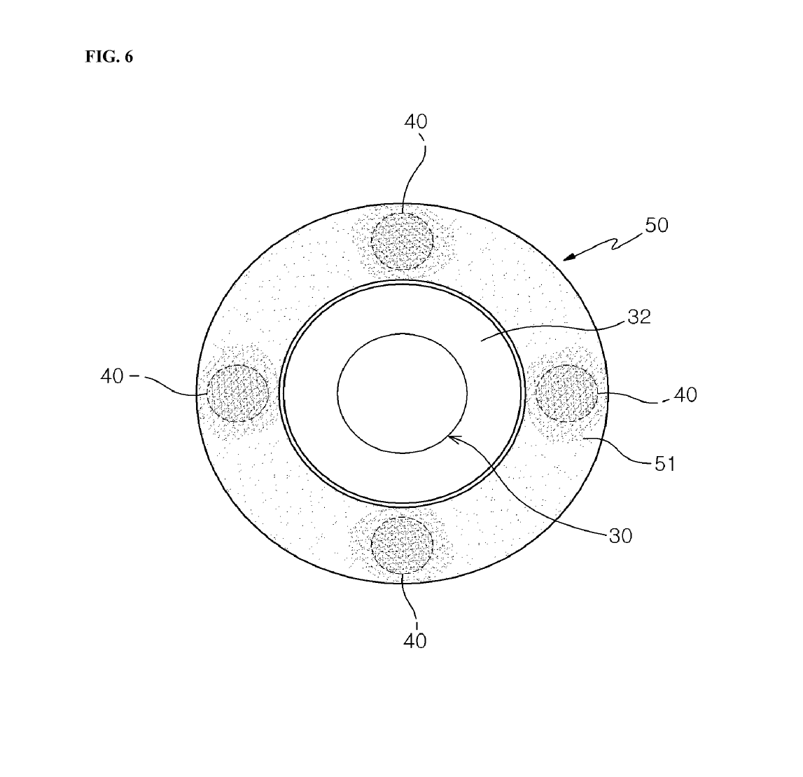

When the power switch (not shown) is turned on, electric power is supplied from the battery or adapter to the components described above, respectively. The micom 38 operates the monitor 37 via the line 37-1 and the LEDs 40 via the output port P1. When the LEDs are turned on, the light is diffused by the convex portion 52 as shown in FIGS. 5 and 6. Although the convex portion 52 diffuses the light with the largest intensity L1 at the center, and with relatively smaller intensities L2 and L3 at the peripheral portions around the center, the lens module 32 receives light summing the intensities L2 and L3 at the front center, which can be calculated by vector operation. Therefore, the LEDs 40 can illuminate an object with uniform illuminance irrespective of the center or peripheral portions of the convex portion 52. It is preferable that the convex portion 52 needs to be designed to have an optimal focus, considering the diameter of the pipe-line body 20.

The light transmission material ring 50 forms patterns 51. Each pattern 51 is larger in a part where an LED exits and gets gradually smaller in a part where an LED does not exist, so that it can illuminate a corresponding object to take videos with uniform illuminance irrespective of whether where an LED exists. The pattern 51 may be formed on the light transmission material ring 50 by attaching a pattern sheet or by being etched.

When the user grips the main body 10 and inserts the pipe-line body 20 into a cavity of a corresponding body that he/she cannot directly view, e.g., the cavity of the nose or ears, the mouth inside, the anus, the eyes, etc., the CCD chip 34 takes videos of the inner cavity, focused by the lens module 32, under the uniform illuminance of the LEDs 40. The AD converter 35 converts the analog video data to digital data. The DSP 36 processes the digital data in the standard image format. The micom 38 outputs the processed image to the monitor 37 via the output port P3. Using the portable skin imaging system, users can directly view a cavity of the body that they want to examine, while carrying it. Therefore, when users detect that a cavity is in an abnormal state with a naked eye via the system, they can take necessary action, compared with the conventional environment where they must directly handle a cavity in an abnormal state with their hand or cotton.

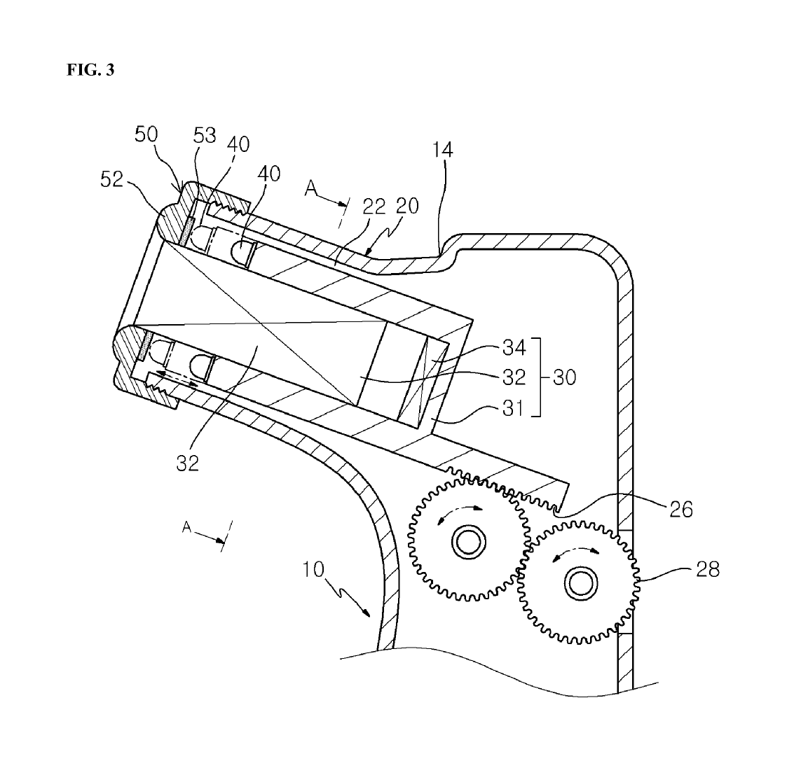

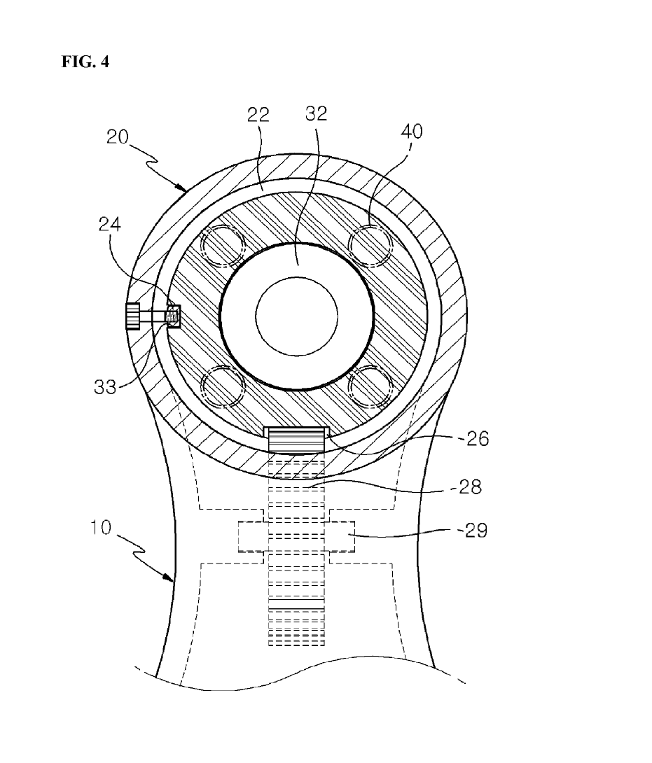

When a first spur gear 28 is rotated in a forward or backward direction to move the lens module 32 and focus on a corresponding object, a second spur gear 28 engaged with the first spur gear 28 is rotated and moves the spur rack gear 26 back and forth as shown in FIGS. 3 to 5. The spur gear 26 is formed on the outer surface of the camera module 30. The focusing operation of the lens module may also be performed in such a way that a small electric motor transfers dynamic force to the spur gears 28. Alternatively, it may also be implemented in such a way that the lens module is equipped with a magnet and the movement is controlled as the magnet is operated via an additional electromagnet. In addition, the video can be enlarged or reduced via an application program in the digital mode.

When the lens module 32 moves back and forth, a base plate 31 forming the CCD camera module 30 also moves in the same direction therewith. The base plate 31 lengthwise forms a guide groove 33 on the outer surface. The guide groove 33 receives a guide 24 fixed to the inner wall 22 of the pipe-line body 20 and prevents the guide 24 from being twisted when the lens module 32 focuses on a corresponding object. The lens module 32 focuses on a corresponding object while moving back and forth, which may be implemented in a sliding mode.

The embodiment of the invention is implemented in such a way that the main body 10 has a larger diameter than the pipe-line body 20, so that only the pipe-line body 20 can be inserted into a cavity of the human body and instead the main body 10 is located at the opening of the cavity. This configuration allows the user to stably examine the inner cavity. The portable skin imaging system is used in a state where only the pipe-line body 20 is inserted into a cavity of the human body. LEDs 40 are sparsely installed to the base plate 31, around the periphery of the lens module 32, and illuminate the inner cavity. The light transmission material ring 50 forms a pattern layer 53 to achieve the uniform illuminance of the LEDs 40 in parts whether LEDs 40 exist or not. The pattern layer 53 forms a pattern in such a way that it is larger in a part where an LED is close to, so that it can limit the transmission of light from the LED, and smaller in a part where an LED does not exist, so that it can allow a large amount of light from the LED to be transmitted. This configuration makes the light transmission material ring 50 transmit light from the LEDs uniformly with respect to the axis.

The light transmission material ring 50 forms a convex portion 52 on the surface, so that the intensity of light can be uniform in the radial direction. In that case, an object receives light from the LEDs 40 with uniform illuminance, and this makes the CCD chip acquire the video in a uniform illuminance state. As shown in FIG. 5, the convex portion 52 emits a relatively strong intensity of light L1 at the center; however, a relatively weak intensity of light L2 and L3 at portions other than the center. The portable skin imaging system according to the invention is designed in such a way that, although the amount of light L2 and L3 is relatively smaller than light L1, since light L2 and L3 is summed in the center space of the light transmission material ring 50, the light intensity is uniform in the radial direction. Therefore, the portable skin imaging system can take videos in the inner cavity with uniform illuminance while the pipe-line body 20 is being inserted in the cavity of the human body and can display the videos on the monitor.

The user grips the main body 10 and inserts the pipe-line body 20 into a cavity of his/her body. The output port P3 is connected to the monitor 37 via the line 37-1 (e.g., I/O interface, not shown). The LEDs 40 illuminates the inner cavity via the light transmission material ring 50 and the CCD camera module 30 takes videos of the inner cavity. The videos are displayed on the monitor 37. When the user puts the pipe-line body 20 into the cavity, the truncated cone-shape portion 12 applies pressure to the opening of the cavity so that he/she can feel the length of pipe-line body 20 to be inserted, and, if the pipe-line body 20 is deeply inserted into the cavity, the stopper step 14 stops the insertion to protect the user's safety.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

The portable skin imaging system for the human body according to the invention can be widely applied to endoscopes that are inserted in and examine the interior of the human body.

1. A portable skin imaging system for the human body, comprising:
a main body (10) shaped to be easily gripped by the hand;
a pipe-like body (20), formed on the top of the main body (10), for putting in the cavity of the human body, the lengthwise axis being inclined against that of the main body (10), and the tip being protruded in the lengthwise direction from the main body (10);
a charge coupled device (CCD) camera module (30) installed inside the pipe-line body (20);
LEDs (40) installed sparsely around the CCD camera module (30); and
a light transmission material ring (50) with a convex portion (52) for uniformly diffusing the light from the LEDs (40).

2. The portable skin imaging system according to claim 1, wherein the light transmission material ring (50) is fixed to the front top of the pipe-line body (20) so that it can be located at the front end of the lens module of the CCD camera module (30).

3. The portable skin imaging system according to claim 1, wherein:
the light transmission material ring (50) forms, in the inside, a pattern layer (53) for limiting the light transmission of light emitted from the LEDS (40); and
the pattern layer (53) increases larger patterns (51) in parts where the LEDs (40) exist and gradually decreases the patterns in parts where the LEDs 40 do not exist.

4. The portable skin imaging system according to claim 1, wherein the CCD camera module (30) comprises:
a lens module (32) shaped a pipe, for adjusting the focus; and
a CCD chip (34) installed inside the lens module (32), for reading video data, and
wherein:
the lens module (32) forms a guide groove (33) on the outer surface in the lengthwise direction, the guide groove (33) guiding the movement of a guide (24) protrudently formed on the inner wall (22) and forms, on the outer surface, a spur rack gear (26) for transferring movement power; and
the spur rack gear (26) is engaged with spur gears (28) one of which is partially exposed from the pipe-line body (20) or the main body (10) and rotated by the user's finger.

5. The portable skin imaging system according to claim 2, wherein:
the light transmission material ring (50) forms, in the inside, a pattern layer (53) for limiting the light transmission of light emitted from the LEDS (40); and
the pattern layer (53) increases larger patterns (51) in parts where the LEDs (40) exist and gradually decreases the patterns in parts where the LEDs 40 do not exist.

6. The portable skin imaging system according to claim 2, wherein the CCD camera module (30) comprises:
a lens module (32) shaped a pipe, for adjusting the focus; and
a CCD chip (34) installed inside the lens module (32), for reading video data, and
wherein:
the lens module (32) forms a guide groove (33) on the outer surface in the lengthwise direction, the guide groove (33) guiding the movement of a guide (24) protrudently formed on the inner wall (22) and forms, on the outer surface, a spur rack gear (26) for transferring movement power; and
the spur rack gear (26) is engaged with spur gears (28) one of which is partially exposed from the pipe-line body (20) or the main body (10) and rotated by the user's finger.
